(19) United States
(12) Patent Application Publication
(10) Pub. No.: US 2003/0083555 A1
(43) Pub. Date: May 1, 2003

(54) Segmented arm support system and method for stabilizing tissue

(76) Inventors: Scott Hunt, Franklin, MA (US); Martin Weinstein, South Dartmouth, MA (US)

Correspondence Address: GENZYME CORPORATION, LEGAL DEPARTMENT, 15 PLEASANT ST CONNECTOR, FRAMINGHAM, MA, 01701-9322, US

(21) Appl. No.: 10/008,509

(22) Filed: Oct. 29, 2001

(51) Int. Cl. A61B 1/32

(52) U.S. Cl. 600/229

(57) ABSTRACT

A segmented arm support system and method for attachment to a surgical retractor includes an arm having a plurality of segments which can be joined together in frictional, locking engagement. In an unlocked state, the flexible arm can be manually positioned and the stabilization device placed over a localized area of tissue. Upon tightening of a cable running through the segments, the segments are brought together in frictional engagement. Each segment has a convex outer wall and a concave inner surface for attachment to adjacent segments. The segments are constructed of a stainless steel substrate material and coated with a high friction plating material to permit frictional engagement of the segments, thereby allowing for adequate tightening and locking of the arm.

### FIELD OF INVENTION

The present invention relates to surgical retractors and devices for stabilizing a predetermined area of the body during a surgical procedure, more particularly to surgical retractors and stabilizing devices used in connection with an improved segmented arm support system that is preferably used in coronary artery bypass grafting surgical procedures, and more specifically to a segmented arm apparatus that is used with various surgical retractors and stabilization devices for use in various surgical procedures.

### BACKGROUND OF THE INVENTION

Diseases of the cardiovascular system affect millions of people each year and are a cause of death for large numbers of people in the United States and throughout the world. A particularly prevalent form of cardiovascular disease involves a reduction in the blood supply to the heart caused by atherosclerosis (coronary artery disease) or other conditions that create a restriction in blood flow at a critical point in the cardiovascular system affecting blood flow to the heart.

One technique for treating such a blockage or restriction is a surgical procedure known as a coronary artery bypass graft procedure, which is more commonly known as “a heart bypass” operation. The surgical correction of occluded or stenosed coronary arteries by means of bypass grafting is among the most common procedures performed today, especially when multiple grafts are needed.

In the coronary artery bypass graft procedure, the surgeon either removes a portion of a vein from another part of the body for grafting or detaches one end of an artery and connects that end past the obstruction in the coronary artery while leaving the other end attached to the arterial supply. When using a vein from another part of the body, the surgeon installs this portion at points that bypass the obstruction. In both cases, the objective is to restore normal blood flow to the heart.

In addition, when using this technique the surgeon makes a long incision down the middle of the chest, saws through the sternum, spreads the two halves of the sternum apart and then performs several procedures necessary to connect the surgical patient to a cardiopulmonary bypass machine to continue the circulation of oxygenated blood to the rest of the body while the heart is stopped and the graft is being sewn in place. Although such a procedure is one common technique for treatment, the procedure is lengthy, traumatic, costly and can damage the heart, the central nervous system, and the blood supply.

Interventional techniques, such as percutaneous transluminal angioplasty (PTCA), have gained popularity as the methods of choice for therapy of atherosclerosis occlusions, for several reasons. The transluminal approach is a minimally invasive technique that subjects the patient to reduced trauma and reduced recovery time, especially when compared to bypass grafts which utilize homologous tissue, such as saphenous vein grafts. Also, the patient often suffers complications at the donor site of the graft that may be worse than the sternotomy and anastomosis.

Although PTCA procedures are often successful, complications can arise, such as restenosis or thrombosis and embolism. Restenosed vessels often require surgical intervention for correction. The surgical correction of restenosis, like the conventional coronary bypass surgical procedure, requires the heart to be stopped and the patient placed on a heart/lung bypass machine during the procedure.

In recent years, and in an effort to reduce cost, risk, and trauma to the patient, physicians have turned to minimally or less invasive surgical approaches to the heart, such as intercostal and endoscopic access to the surgical site. With such procedures, the heart is beating during the surgical procedure. Thus, there is no need for any form of cardiopulmonary bypass, and there is no need to perform the extensive surgical procedures necessary to connect the patient to such a bypass machine.

Such attempts at performing minimally invasive bypass grafting on a beating heart, however, have been characterized as tedious, dangerous and difficult because of the delicate nature of the surgical procedure, the lack of adequate access through a reduced surgical field, and the lack of an ability to adequately stabilize and reduce tissue movement at the graft site. Because these procedures are performed while the heart muscle continues to beat, the blood continues flowing and the heart continues moving in three-dimensional movement while the surgeon attempts to sew the graft in place. Also, the surgical procedure to install the graft requires placing a series of sutures through an extremely small vessel and onto tissue that continues to move during the procedure. It is necessary that these sutures be fully and securely placed so that the graft is held firmly in position and does not leak.

There is disclosed in U.S. Pat. No. 5,730,757 an access platform for the dissection of an internal mammary artery. The described access platform has first and second blades interconnected to a spreader member that laterally drives the blades apart or together, and support pads interconnected to the first blade. A torsional member is operably interconnected to the first blade and the spreader member, and is used to vertically displace the first blade in either direction, thus increasing the surgeon's working space and visual access for dissection of the internal mammary artery. A tissue retractor interconnected to the blades is used to draw the soft tissue around the incision away from the surgeon's work area. It is further provided that the access platform can include a port that can be used to mount a heart stabilizer instrument.

There also is described in U.S. Pat. No. 5,875,782 granted to Ferrari et al.; U.S. Pat. No. 6,033,362 granted to Cohn; U.S. Pat. No. 6,102,854 granted to Cartier et al.; and U.S. Pat. No. 5,894,843 granted to Benetti et al. various devices for stabilizing a predetermined area on the heart or other organ of a patient, e.g., to enable a surgical procedure on the beating heart. These devices include various stabilization members and an elongated arm. The arm segments can be movably attached to a rib retractor so that a person is not required to hold the arm segment.

There also is described in U.S. Pat. No. 5,836,311 granted to Borst et al. an apparatus for stabilizing a predetermined area on the heart or other organ of a patient, e.g., to enable a surgical procedure on the beating heart. The apparatus includes a single legged or bifurcated member having a plurality of suction members thereon which are attached to the surface of the heart using suction pressure. The arm portion of this device can be movably attached to a rib retractor or other surgical device so a person is not required to hold the arm segment and the suction device may be locked into position against the surface of the heart.

U.S. Pat. No. 5,947,896 granted to Sherts et al. describes an articulating arm made up of segments/link members. In one particular embodiment therein (see FIGS. 41-49), an articulated arm is disclosed having a cable passing through link members and an “over-center” locking mechanism for tightening the cable. As is known to those skilled in the art, in such an articulating arm, the proximal link members are subjected to larger torques than more distal link members, due to large forces applied at the distal end that produce moment arms at the proximal end. Because the link members have identical dimensions and there is no mechanism provided to handle the additional torque acting on the link members at the proximal end, it is apparent that the proximal link members are not capable of absorbing the full amount of torque that is present upon tightening the cable. Accordingly, the degree of tightening that can be achieved in Sherts et al. is limited, and adequate stabilization cannot be assured. Moreover, in Sherts et al., only a small number of link members are provided, limiting the range of movement of the articulated arm.

U.S. Pat. No. 5,899,425 to Corey, Jr. et al. describes a stabilizer with a flexible arm having a series of elements of progressively decreasing size from the proximal end to the distal end of the arm. Because larger elements are used at the proximal end, where the elements are subjected to larger torques, the arm is able to resist articulating movement in the locked state. However, use of the larger elements reduces flexibility in the arm in the unlocked state. Therefore, the range of movement and the available locking positions are limited.

It is therefore desirable to provide a new system and devices related thereto for stabilizing a predetermined area of an organ such as the heart and methods for using such devices. It is particularly desirable to provide an articulating arm with sufficient flexibility to allow a large range of motion and a wide variety of positions, but one that is capable of adequately locking to assure stabilization during surgical procedures.

### SUMMARY OF THE INVENTION

The present invention features a system for retracting, stabilizing or manipulating a predetermined area of the body. The system includes a segmented arm system or apparatus and a tissue support or stabilization device, and methods of use related thereto.

The segmented arm system and related devices and apparatuses that are featured herein are particularly advantageous for use in performing off-pump coronary artery bypass grafting procedures in which the heart remains beating during the surgical procedure and/or valve surgery where the heart is stopped. One advantage of the present invention relates to the versatile use of the segmented arm system which is connected to an arm or rack section of the retractor and also retains a stabilization device or surgical implement in a desired position. The use of the external rail system on the retractor allows a segmented arm apparatus to be attached to the retractor at any desired location and does not require that the segmented arm apparatus be slid on from an end of an arm or specially attached in certain specific locations. Additionally, the segmented arm system of the present invention allows for a full range of three-dimensional motion of the stabilization device or surgical instrument which is controlled by a single knob that is spaced apart from each of the retractor and the segmented arm apparatus. The segmented arm apparatus is also easily and conveniently manipulated by the surgeon and is movable so as not to obstruct the surgeon's view of the desired target location.

The stabilization device preferably includes devices of the type commonly known as the Cohn Cardiac Stabilizer or the Immobilizer marketed by the Genzyme Corporation of Cambridge, Mass., although horseshoe or suction type devices may also be used. The preferred form of the stabilization device is a generally square, rectangular, or teardrop shaped member having a planar surface with centrally located opening therein. This opening is the area through which the surgeon performs the anastomosis or other procedure on the tissue of the beating heart. The stabilization device is preferably a multiple piece member so that once the anastomosis is completed, the pieces or an end portion thereof may be separated to remove the device from around the anastomosis. Flexible tapes can be sutured through the tissue and then threaded through the stabilizing device to provide temporary vessel occlusion. Once the stabilization device is positioned in the desired orientation and location in contact with the tissue, the flexible tapes are then pulled snug through the opening of the stabilization device to provide a system that captures the predetermined area of the tissue.

The segmented arm system preferably includes an elongated articulating arm having a proximal mounting assembly for attachment to the retractor and a distal connector thereon for releasably connecting the stabilization device or surgical instrument to the articulating arm. The distal connector allows the stabilization device to be pivotally and slidably moved to a desired position into contact with the predetermined area of the tissue of the patient. The segmented arm system of this embodiment preferably includes a plurality of segments positioned along the length of the arm. The segments provide a plurality of locations for relative movement of the stabilization device as well as providing locations for fixing the desired position of the stabilization arm system along the retractor and relative to the stabilization device.

Additionally, the movable segments allow the user to position at least a portion of the plurality of arm segments away from the desired surgical site so that the articulating arm does not obstruct the view of the surgeon or the assistant while providing sufficient leverage to provide a stable surgical site and to allow access to various locations on the heart of the patient. The segments each have approximately the same size and shape, are of low profile, and are capable of a large range of movement, allowing the articulating arm to be manually arranged in a desired position. Each segment preferably is made of a material with high stiffness (e.g., stainless steel) coated with a high friction material (e.g., nickel, gold, silver, tin, copper, or an elastomer).

Other aspects and embodiments of the invention are more fully discussed below.

### BRIEF DESCRIPTION OF THE DRAWING

For a fuller understanding of the nature and desired objects of the present invention, reference is made to the following detailed description taken in conjunction with the accompanying drawing figures wherein like reference character denote corresponding parts throughout the several views and wherein:

FIG. 1 is a perspective view showing a segmented arm apparatus of the present invention;

FIG. 2 is a partial exploded parts view of the segmented arm apparatus of FIG. 1, including the tightening assembly, mounting assembly, and one segment;

FIG. 3 is a sectional view showing details of the tightening assembly and mounting assembly of the segmented arm apparatus;

FIG. 4 is a partial exploded parts view of the distal assembly of the segmented arm apparatus;

FIG. 5 is a sectional view showing details of the attachment of the stabilization device at the distal end of the segmented arm apparatus;

FIG. 6 is an enlarged sectional view showing details of a plurality of segments of the segmented arm apparatus;

FIG. 7 is a perspective view of the segments depicted in FIG. 6; and

FIG. 8 is a cross-sectional view of one of the segments depicted in FIGS. 6 and 7.

### DETAILED DESCRIPTION OF THE INVENTION

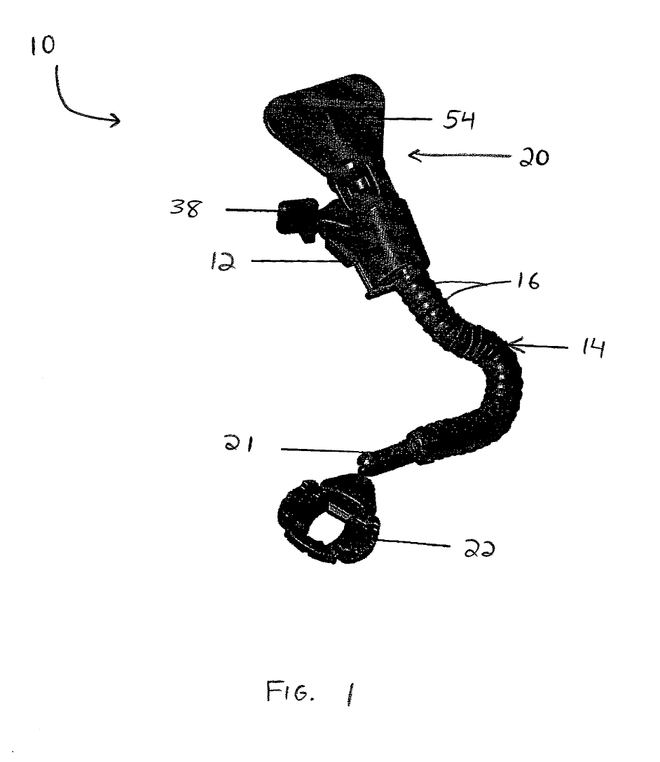

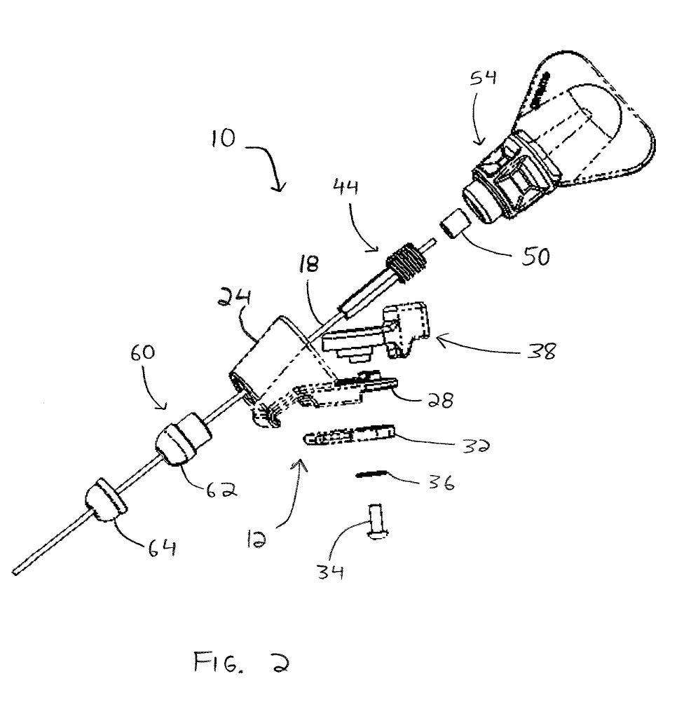

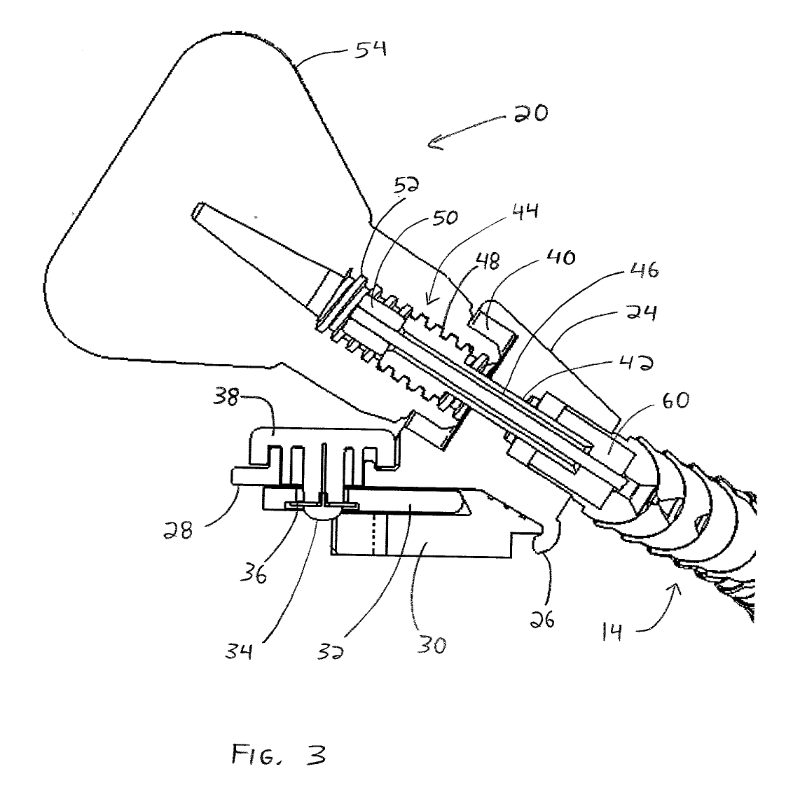

FIG. 1 illustrates an example of a segmented arm apparatus 10 of the present invention. The segmented arm apparatus 10 includes a proximal mounting assembly 12 for attaching the apparatus to a surgical retractor (see FIG. 3). An elongated articulating arm 14 made up of a plurality of segments 16 is connected to the mounting assembly 12 at a distal end thereof, the articulating arm having a cable 18 (see, e.g., FIG. 2) received in a passage extending through approximately the middle of each segment. A tightening assembly 20 is attached to the proximal end of the mounting assembly 12 and connected to the cable 18 for tightening the cable and thus forcing the segments 16 of the articulating arm 14 into locking, frictional engagement.

Attached to the distal end of the articulating arm 14 is a distal connector 21 for receiving a stabilization device 22, e.g., a tissue stabilizer or other surgical instrument. The stabilization device 22 can fix or immobilize a section of tissue of an organ, such as the heart wall, when the articulating arm 14 is in a locked condition, for use in performing surgical operations.

As shown in FIGS. 2 and 3, the mounting assembly 12 includes a mounting block 24 having a lip 26 and a mounting extension 28 to permit mounting of the segmented arm apparatus 10 on a retractor 30. Details of the retractor and attachment of the segmented arm apparatus to the retractor is provided in U.S. Ser. No. 09/746,310, filed on Dec. 20, 2000, which application is incorporated by reference herein.

The mounting assembly 12 of the present invention includes a cam 32 attached to the mounting block 24 by a threaded fastener or screw 34. The screw 34 preferably receives a washer 36 to connect with a surface of the cam 32, and the screw is threaded into a lever 38, with the mounting extension 28 being sandwiched between the cam 32 and lever 38. Because the lever is in contact with the cam 32 by an eccentric boss of the lever (not shown), rotation of the lever causes the cam 32 to move within mounting extension 28. By rotating the lever between first and second positions, the cam can be adjusted laterally on a plane parallel to the mounting extension 28, such that the cam moves toward or away from the retractor 30. Thus, the mounting assembly of the present invention provides a simple arrangement for attaching the segmented arm apparatus to a retractor, allowing a surgeon to position and reposition the segmented arm apparatus as desired.

The tightening assembly 20 is received in a bore 40 formed in the proximal end of the mounting block 24. The bore 40 extends distally to form a narrow slot 42 for receiving therein a shaft portion 46 of an adjustment element 44. The shaft portion 46 can be formed in a shape corresponding to the shape of the slot 42. For example, the shaft can have a hexagonal shape to fit within a hexagonally-shaped slot 42. Extending in a proximal direction from the bore 40, the adjustment element 44 includes a threaded portion 48 that is generally of larger diameter than the shaft portion 46, so that when the shaft portion 46 is engaged in the slot 42, the threaded portion 48 remains positioned outside of the slot. The cable 18 extends through the adjustment element 44 and terminates beyond the proximal end of the adjustment element 44 with a crimp 50 preferably made of stainless steel or similar material. The adjustment element 44 and crimp 50 can be received within the adjustment knob 54. The knob 54 contains a threaded fastener portion 52 adapted to engage the threaded portion 48 of the adjustment element 44. Therefore, the adjustment element 44 and crimp 50 are received within the opening of the knob, and upon rotation of the knob, the crimp 50 and the cable can be tightened or released. The mechanism for tightening the cable and thereby forcing the segments of the articulating arm into locking, frictional engagement is discussed further below.

With reference to FIG. 2, a mounting socket 60 is secured to the distal end of the mounting block 24 by threaded or non-threaded engagement with the mounting block 24. The mounting socket 60 includes a lower (distal) surface 62 formed in a convex, generally spherical/cylindrical or crowned shape. The convex surface of the mounting socket is adapted to be received in a concave surface of a first segment 64 of the articulating arm 14. The first segment 64 also is referred to herein as the most proximal segment of the arm 14. Other segments 16 of the articulating arm 14 are connected in a similar ball-and-socket manner.

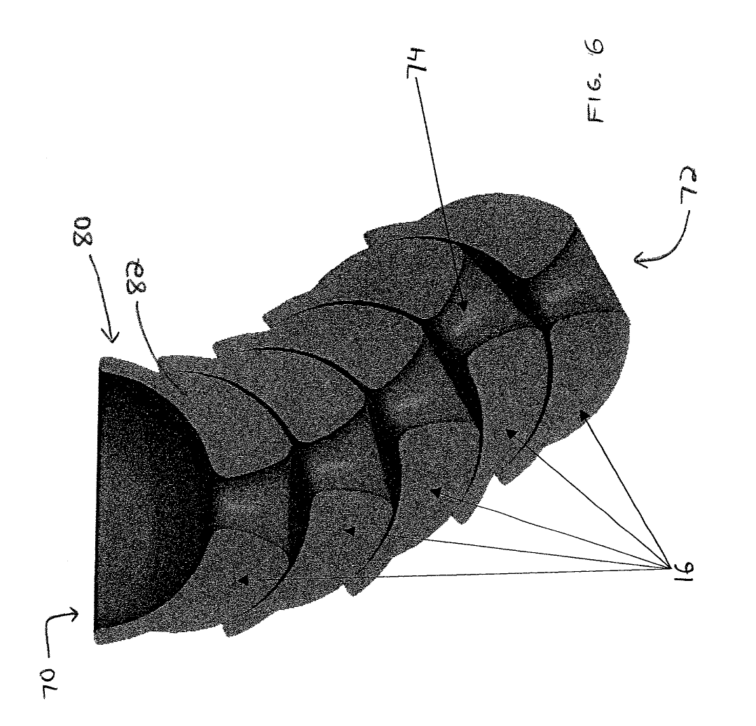

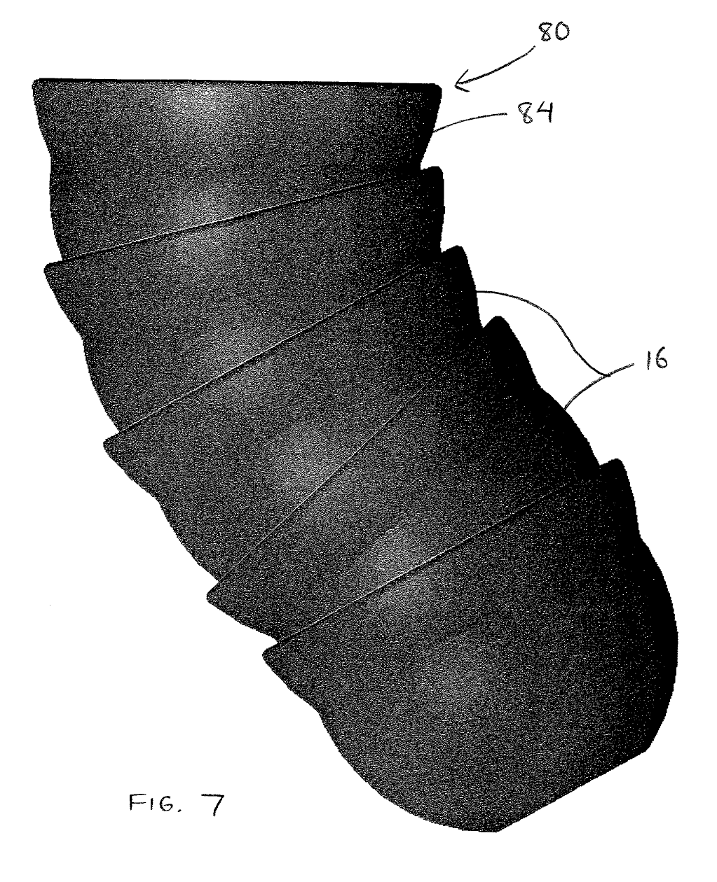

As seen in FIG. 1, the articulating arm 14 includes a plurality of segments 16 each having approximately the same size and shape, adjacent segments being joined together in a ball-and-socket type configuration. Selected segments 16 of the articulating arm 14 are shown in greater detail in FIGS. 6 and 7. Each segment 16 includes a convex outer surface located at one end of the segment, shown herein as the distal end 72, and a concave inner surface oriented in the opposite direction, i.e. opening toward the proximal end 70. Thus, an outer surface (ball shape) of each segment 14 is configured and arranged in a mating relationship with an inner surface (socket shape) of an adjacent segment. Each segment 16 further includes an internal passage 74 for accommodating the cable 18.

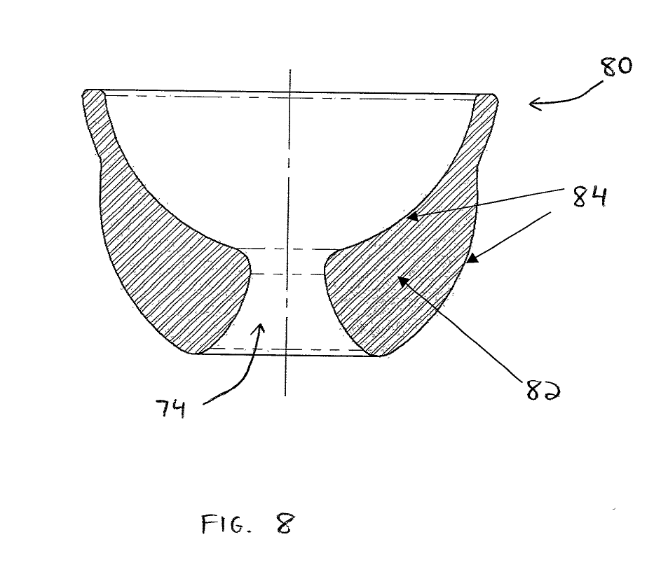

The articulating arm 14 preferably includes approximately 20 to 40 segments, though more or fewer segments are also within the scope of the invention. In the example shown in FIG. 1, the arm includes 28 segments. Each segment preferably constitutes a substrate made of a material of high stiffness, preferably stainless steel, that is covered with a high friction plating material. For example, as seen in FIGS. 6 to 8, segment 80 has a substrate 82 made of stainless steel and an outer plating material 84 covering the substrate A preferred plating material is nickel, although other metals can also be used in accordance with the invention, including but not limited to: gold, silver, tin, and copper. The plating material also can be formed of non-metal elastomers.

By manually rotating the adjustment knob 54 in a clockwise direction (looking distally from the knob toward the stabilization device), a pulling force is exerted on the cable 18, causing the cable to be placed under tension and thereby tightened. By tightening the cable, the segments 16 of the articulating arm 14 become locked together as a result of frictional forces exerted between connected segments. Segments closer to the proximal end of the articulating arm experience larger torques due to the larger forces applied to the distal end of the segmented arm apparatus 10.

In prior art segmented arm devices, in order to assure proper engagement of the segments, segments at the proximal end were sized larger than segments at the distal end. Such an arrangement is effective for producing locking engagement of the segments, but is undesirable for use in the present invention due to the large profile of segments located near the proximal end and the reduced flexibility of the articulating arm due to the larger segments.

Therefore, in accordance with the present invention, and in order to prevent slippage and assure adequate locking engagement of the segments upon tightening of the cable, the segments are coated with a high friction plating material so that the segments frictionally engage each other upon tightening of the cable. An example of a hard, high friction plating material is nickel. An example of a soft, high friction plating material is an elastomer. When the segments are coated with a soft plating material, there is a prescribed amount of “bite” exhibited in adjacent segments upon tightening of the cable and engagement of the segments. This bite results from impact of the softer coating of adjacent segments, whereby the coatings of adjacent segments engage and become compressed against the harder underlying stainless steel substrates, allowing the plating material of adjacent segments to “bite” or be compressed together, and thereby leading to a tighter frictional engagement for obtaining sufficient locking of the segments at both the distal and proximal ends of the articulating arm. The segments still maintain an overall rigidity and stability due to the strength of the substrate stainless steel material present in each segment.

By including a plurality of segments coated with a high friction material, the segments can be manufactured of approximately equal size. It is not necessary to include larger proximal segments, since the larger torques experienced at the proximal end of the segmented arm apparatus are counteracted by frictional, locking engagement of the segments. In the case of segments coated with a soft, high friction plating material, the plating material tends to compress against the harder stainless steel substrate. Therefore, the articulating arm 14 can be comprised of smaller, lower profile segments, as compared with prior art devices.

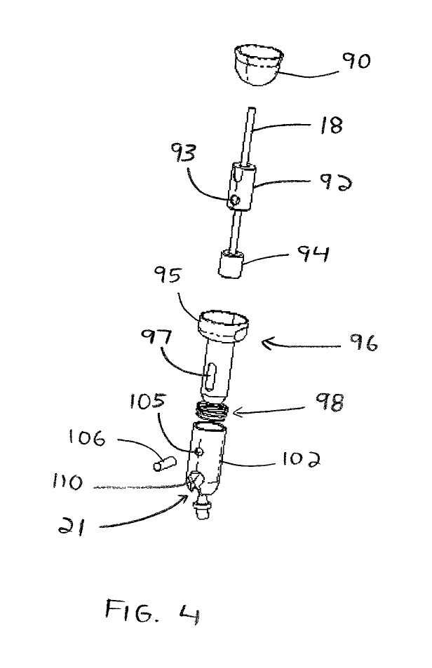

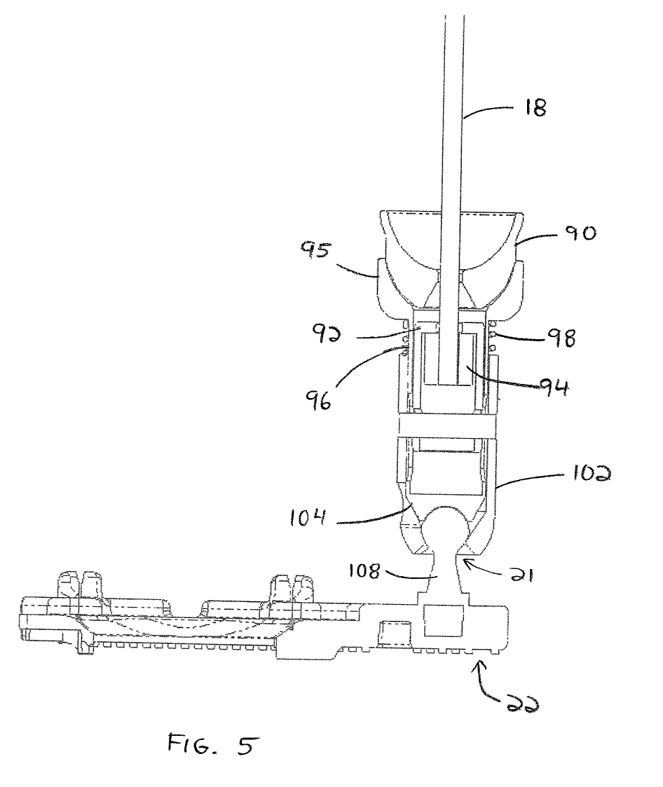

FIGS. 4 and 5 illustrate a distal end of the segmented arm apparatus including the most distal segment 90 and the cable 18 extending through a passage in the segment 90. The most distal segment 90 is received in a basin 95 formed in a proximal portion of a plunger 96, the basin 95 having a generally concave surface to compliment the convex surface on the outside of the segment 90. The distal end of the cable 18 terminates with a crimp 94 preferably made of stainless steel or similar material. The crimp 94 is received in a cable receptacle 92 which is accommodated within the plunger 96.

The plunger 96 is slidably disposed in a socket 102, whereby a portion of the plunger 96 extending distally from the basin 95 is biased against a proximal portion of the socket 102 by a spring 98, allowing the socket 102 to be compressed toward the basin 95. A pin 106 extends through a slot 97 in the plunger 96 and a hole 93 in the cable receptacle 92, and attaches to either side of the socket via hole 105, thereby connecting the plunger 96, cable 18, and socket 102. This type of attachment allows for longitudinal movement of the plunger within the socket.

The distal connector 21 is formed at a distal end of the socket 102 and plunger 96, the plunger 96 including a socket portion 104 preferably configured and arranged to receive a post 108 attached to the stabilization device 22. The post 108 and distal connector 21 form a ball-and-socket connection, such that upon loosening of the adjustment knob 54, the post 108 and thus the stabilization device 22 can be released from the segmented arm assembly through an opening 110 in a section of the socket 102. Accordingly, the stabilization device 22 can be replaced easily and substituted for on the segmented arm apparatus.

Various modifications and additions can be made to the herein described invention. For example, the articulating arm 14 preferably is covered with an outer sleeve made of a thermoelastomer or other biocompatible material, thereby protecting the patient from exposure to the plating material.

Further modifications can include a force-releasing mechanism or mechanical stop positioned inside the knob to measure the force applied to the cable and prevent further rotation of the knob if the applied force exceeds a prescribed threshold.

A low-pitch knob thread design (e.g., a turnbuckle-type mechanism with left- and right-handed concentric threads) can be implemented to provide a larger axial movement of the cable in response to each turn. Additional modifications to the knob design can include a drum with pawl and lever, a cam with a spring-loaded cable, or a three-bar linkage arrangement.

Although a preferred embodiment of the invention has been described using specific terms, such description is for illustrative purposes only, and it is to be understood that changes and variations may be made without departing from the spirit or scope of the following claims.

What is claimed is:

1. A segmented arm support apparatus for attachment to a surgical retractor, comprising:
an articulating arm having a plurality of segments, each segment having an outer wall and an inner surface with a passage extending through each segment, the outer wall of a first segment in mating relationship with the inner surface of a second segment, each segment being formed of a material with high stiffness coated with a high friction plating material;
a cable extending through the passage of each segment;
a device for tightening the cable, thereby causing the mating segments to be brought into tight frictional engagement and compressing the plating material; and
a tissue stabilization device attached to the articulating arm, the stabilization device being lockable upon tightening the cable.

2. The segmented arm support apparatus of claim 1, wherein each segment is defined by a convex outer wall and a concave inner surface.

3. The segmented arm support apparatus of claim 2, wherein the convex outer wall of the first segment engages the concave inner surface of the second segment.

4. The segmented arm support apparatus of claim 1, wherein the material with high stiffness comprises stainless steel.

5. The segmented arm support apparatus of claim 1, wherein the mating relationship exists between each segment.

6. The segmented arm support apparatus of claim 1, wherein the high friction plating material is softer than the material with high stiffness.

7. The segmented arm support apparatus of claim 1, wherein the high friction plating material is selected from the group consisting of nickel, gold, silver, copper, tin, and an elastomer.

8. The segmented arm support apparatus of claim 1, wherein the high friction plating material comprises nickel.

9. The segmented arm support apparatus of claim 1, wherein during tightening of the cable, the high friction plating material of adjacent segments frictionally engages.

10. The segmented arm support apparatus of claim 1, wherein the stabilization device is removably attached to the segmented arm support apparatus.

11. The segmented arm support apparatus of claim 10, and further including a movable socket to receive the stabilization device.

12. The segmented arm support apparatus of claim 11, wherein the movable socket is slidable along a plunger attached to an end of the cable.

13. The segmented arm support apparatus of claim 12, wherein the movable socket is biased against the plunger by a spring.

14. The segmented arm support apparatus of claim 1, and further including a mounting block for attachment to a retractor.

15. The segmented arm support apparatus of claim 14, the mounting block including a lever for positioning a cam to engage the retractor.

16. A method for stabilizing a localized area of tissue on a patient, comprising the steps of:
providing a segmented arm support apparatus including an articulating arm with a plurality of segments, each segment having an outer wall, an inner surface, and a passage for receiving a cable, the outer wall of a first segment in mating relationship with the inner surface of a second segment, each segment being formed of a material of high stiffness coated with a plating material;
providing a stabilization device attached to the articulating arm; and
tightening the cable so that the plating material of the first and second segments frictionally engages, thereby causing the stabilization device to lock.

17. The method of claim 16, wherein the material of high stiffness comprises stainless steel.

18. The method of claim 16, wherein the plating material is selected from the group consisting of nickel, gold, silver, copper, tin, and an elastomer.

19. The method of claim 16, wherein the plating material comprises nickel.

20. The method of claim 16, wherein the stabilization device is removably attached to a socket in the articulating arm.

21. The method of claim 16, and further including a step of replacing the stabilization device by loosening the cable and removing the stabilization device from a socket.

22. The method of claim 21, wherein the socket is biased against a plunger housing an end of the cable.

23. The method of claim 16, and further including a step of mounting the segmented arm support apparatus on a retractor.

24. The method of claim 23, wherein the mounting step includes manually rotating a lever connected with a cam that attaches to the retractor.
